(19) United States
(12) Patent Application Publication
(10) Pub. No.: US 2002/0055684 A1
(43) Pub. Date: May 9, 2002

(54) Two-headed focusing stethoscope (THFS)

(76) Inventor: Steven Craig Patterson, Middleborough, MA (US)

Correspondence Address: Steven C. Patterson, 405 Marion Road, Middleborough, MA, 02346, US

(21) Appl. No.: 10/011,862

(22) Filed: Oct. 22, 2001

Related U.S. Application Data
(60) Provisional application No. 60/244,706, filed on Oct. 31, 2000.

(51) Int. Cl. A61B 5/02

(52) U.S. Cl. 600/528; 600/526

(57) ABSTRACT

An electronic stethoscope having two listening heads, functionally equivalent to one another, each containing a microphone. The signal of one microphone is subtracted from the signal of the other microphone, and the resulting difference signal is amplified, using either analog or digital signal processing techniques. The amplified difference signal is transduced to sound by headphones. Sounds which originate in the plane equidistant from both microphones (the “midplane”) are eliminated altogether, a fact which may be exploited to dissect a composite sound into two component sounds. Other sounds are attenuated in a predictable pattern, producing a focusing effect which increases as the microphones are brought closer together. The stethoscope incorporates features which minimize electromagnetic interference, enhance low-frequency throughput, reject amplified Johnson noise, and conserve battery power.

### FEDERALLY SPONSORED RESEARCH

Not applicable

### SEQUENCE LISTING OR PROGRAM

Not applicable

### BACKGROUND OF THE INVENTION

1. Field of Invention

This invention relates to the field of stethoscopes, more specifically to noise-suppressing electronic stethoscopes.

2. Description of Prior Art

### Air Column Stethoscopes

Conceived by René Laënnec in 1816, the acoustic air column stethoscope was originally a rigid, monaural device. Dr. George Cammann introduced a binaural model, similar in design to today's stethoscopes, in 1852. Dr. Howard Sprague added a selectable bell/diaphragm combination in 1926. A lightweight model, the popular Littman, was introduced in 1961.

The simple design of the acoustic stethoscope provides some amplification and sound selectivity. Vibrations of the underlying skin produce waves of alternating compression and rarefaction in the air inside the chest piece. Funneled through the tubing, the sound waves impinge upon the ear drums, where they are, in effect, amplified. The instrument's ear tips block most environmental sound.

The air column stethoscope has its shortcomings, however. Sounds transmitted through tissue undergo both frequency distortion and attenuation. The corrupted physiologic sounds which reach the chest piece are, furthermore, a mixture of those under study (e.g. heart sounds), and those not under study (e.g. lung and bowel sounds). Environmental sounds, transmitted both through the body being examined and through the instrument itself, further adulterate the sounds of interest. Frequency-weighted dissipation of sounds in the tubing adds still more distortion.

It is frequently necessary for the practitioner to characterize a particular, often subtle, corrupted physiologic sound amidst a din of louder contaminating sounds. This common situation challenges even the most skilful physicians, and far too often ends in deferred diagnoses or diagnostic errors. Because of these shortcomings, the medical community sought improved means of auscultating physiologic sounds.

### Electronic Stethoscopes

An “electric” stethoscope was developed by S. G. Brown in 1910, but the idea languished for more than 50 years. Interest in electronic stethoscopes, which utilize a transducer to convert air pressure variations into electronic signals, revived in the 1960s. Early models provided amplification, but their uneven frequency response interfered with the faithful reproduction of physiologic sounds. Later models added low-pass or band-pass filters, with corner frequencies selected to pass most physiologic sounds while attenuating some environmental sounds.

Newer models are designed to actually delete environmental sounds. Some, such as that described in U.S. Pat. No. 6,028,942 to Greenberger, use a single noise-subtracting transducer, with a diaphragm which is exposed to a combination of physiologic and contaminating environmental sounds on one side, and directly to the environment on the other. The net sound pressure on the diaphragm is converted to an electrical impulse.

More typically, prior art noise-reducing stethoscopes, such as those described in U.S. Pat. No. 5,492,129 to Greenberger, incorporate two transducers (FIG. 1). One transducer is enclosed in the stethoscope's chest piece, and transduces both physiologic (P) and contaminating environmental sounds. Contaminating environmental sound enters the “physiologic” sound transducer both through the chest piece (EC) and through the patient's body (EB). The difference in the speed of sound in the two media results in phase shifts. In addition, each medium introduces a characteristic pattern of frequency distortion. Environmental sound distorted by its passage through the chest piece differs, therefore, from environmental sound distorted by its passage through flesh.

The other transducer is open to the environment. It transduces environmental sound which reaches it directly (ED), in addition to some transmitted through its housing (EC). If the housing is in contact with the patient, a small component of physiologic sound (P), and some environmental sound which has been transmitted through the patient's body (EB), is also transduced.

It is desirable that the “environmental” transducer signal to be subtracted from the “physiologic” transducer signal quantitatively match the environmental contamination therein. In some embodiments of the prior art, this is accomplished by simply limiting the access of ambient noise to the “environmental” transducer. Others (e.g. that of U.S. Pat. No. 5,492,129 to Greenberger) multiply the “environmental” transducer signal by a fixed gain factor (f<1) to match its environmental component (ED+EC+EB) quantitatively with that (EC+EB) of the “physiologic” transducer signal. (One implementation, described in U.S. Pat. Nos. 5,539,831 and 5,610,987, both to Harley, adds a third transducer, and uses digital signal processing to achieve a more exact match.) The gain-adjusted “environmental” transducer signal is then subtracted from the “physiologic” transducer signal.

This approach fails to recognize that direct environmental sound (ED) and its indirect progeny (EC and EB) differ in phase and frequency distribution. Subtraction reduces each of the components of the “physiologic” transducer signal by its gain-adjusted counterpart from the “environmental” transducer signal. The difference signal presented to the user is contaminated by an inverted version of the direct environmental component of the “environmental” transducer signal [ED (INV.)].

Fortunately, the three environmental sound components share some commonalities. If this were not so, the sound presented to the listener would contain even more environmental contamination than the original “physiologic” transducer signal (as diagrammed). The arrows in FIG. 1 indicate that the inverted direct environmental signal component [ED (INV.)] partially cancels the indirect environmental sound signals (EC and EB). The net effect of subtraction is therefore a reduction in environmental noise.

### Problems Identified in the Prior Art

Despite improvements in the prior art, several problems remain:

(a.) The prior art primarily addresses reducing or removing sounds of environmental origin. Filters are used to attenuate certain frequencies, but they do not discriminate between environmental sounds, physiologic sounds originating at the site under study, and physiologic sounds originating at other sites. Even with these improvements, it is left to the practitioner to identify and assess subtle signs of disease hidden amidst other, often considerably louder, physiologic sounds in the pass band.

(b.) In order to focus on a physiologic sound of interest, the user of a prior art stethoscope must move the chest piece close to the source of the sound, and away from competing sound sources. This technique relies entirely on the attenuation of sound with distance, and is ineffective when competing sound sources are juxtaposed.

(c.) Prior art noise-suppressing electronic stethoscopes use a capacitor to couple each transducer to its signal processing circuitry. The capacitor acts as a gatekeeper, blocking the transducer's direct current bias, while passing its changing alternating current signal. Unfortunately, capacitors attenuate signal components of very low frequency. Many sounds traditionally evaluated by acoustic stethoscopy are vulnerable to such attenuation; the dominant frequency of the first heart sound, for example, is just 22.6±9.6 Hz.

(d.) Prior art electronic stethoscopes have switches which must be deliberately set to the ‘on’ position before the device may be used. This is inconvenient, as a typical practitioner uses his/her stethoscope dozens of times each day. The battery begins to drain when the device is switched on, and continues to drain until it is switched off, whether it is in use or not. Allowing the battery to drain when the device is not in use shortens battery life.

(e.) The signal processing circuitry of prior art noise-suppressing electronic stethoscopes is unnecessarily complicated, resulting in increased manufacturing costs.

### BRIEF SUMMARY OF THE INVENTION

### Objects and Advantages

Accordingly, several objects and advantages of the present invention are:

(a.) to enable the user to focus his/her attention on a physiologic sound of interest, by subtracting contaminating environmental and physiologic sounds;

(b.) to enable the user to listen to the sounds of two juxtaposed sound sources, one at a time;

(c.) to preserve clinically-important sounds of very low frequency;

(d.) to provide a means of automatically powering an electronic stethoscope only when it is in use, thus conserving battery power;

(e.) to accomplish the foregoing objects with substantially less complex signal-processing circuitry than existing noise-suppressing electronic stethoscopes.

Further objects and advantages of my invention will become apparent from a consideration of the drawings and ensuing description.

### Summary

The present invention is an electronic noise-suppressing stethoscope comprised of two identical chest pieces, each shielding a transducer from environmental noise, which are placed within a few inches of one another on the object of study. The signals of the transducers are processed by electronic circuitry to produce an amplified difference signal, which is transduced to sound by speakers.

Subtraction of the signals of the two proximate transducers results in a focusing effect. Sounds which originate close to either transducer are preserved, while both environmental and physiologic sounds which originate farther away are sharply attenuated. Sounds equidistant from the transducers are eliminated altogether, a feature which enables the user to listen to the sounds of two juxtaposed sound sources, one at a tine.

The novel subtraction strategy implemented in the stethoscope obviates the need for capacitors prior to filtering, preventing attenuation of clinically-important low-frequency sounds due to series capacitance. Momentary-on switches close the circuit to the stethoscope's battery only when the chest pieces are pressed against an object of study, conserving battery power. The stethoscope's electronic circuitry is uncomplicated, allowing for mass production at reasonable cost.

### BRIEF DESCRIPTION OF THE DRAWINGS

FIG. 1 is a diagrammatic comparison of the subtraction strategies employed by the prior art and the present invention.

FIG. 2 is a pictorial view of the two-headed focusing stethoscope in use.

FIG. 3 is a 3-dimensional graphical representation of the relative strength R of difference signals resulting from sounds of equal intensity originating in the same plane as transducers A and B, when transducer A is at (−0.5,0) and transducer B is at (+0.5,0).

FIG. 4 is a logarithmic contour plot of the strength of difference signals resulting from sounds of equal intensity originating in the same plane as transducers A and B, when transducer A is at (−0.5,0) and transducer B is at (+0.5,0).

FIG. 5 is a graph comparing the focusing performance of the THFS to the attenuation of sound due to distance alone, when x_{S}=x_{A}.

FIG. 6 is a logarithmic contour plot of the focusing performance of the THFS compared to that of a single-transducer acoustic or electronic stethoscope.

FIG. 7 is a block diagram depicting signal processing in a simple analog version of the THFS.

FIG. 8 is an electrical schematic diagram of a simple analog version of the THFS.

FIG. 9 is an electrical schematic diagram of a transistor-based power amplifier suitable for use with any of several possible embodiments of the invention.

FIG. 10 is an electrical schematic diagram of an integrated circuit-based power amplifier suitable for use with any of several possible embodiments of the invention.

FIG. 11 is a cutaway side projection of the chest piece of the THFS.

FIG. 12 is a cutaway front projection of the chest piece of the THFS.

FIG. 13 is a modified block diagram detailing signal processing in the preferred analog version of the THFS.

FIG. 14 is an electrical schematic diagram of the preferred analog version of the THFS.

FIG. 15 is a block diagram depicting signal processing in the digital version of the THFS.

FIG. 16 is an electrical connections diagram of the power-up circuitry of a digital version of the THFS.

FIG. 17 is a logic and electrical connections diagram of the signal processing portion of a digital version of the THFS.

FIG. 18 is a timing diagram of a digital version of the THFS.

### DETAILED DESCRIPTION OF THE INVENTION

### Conceptual Basis of the Invention

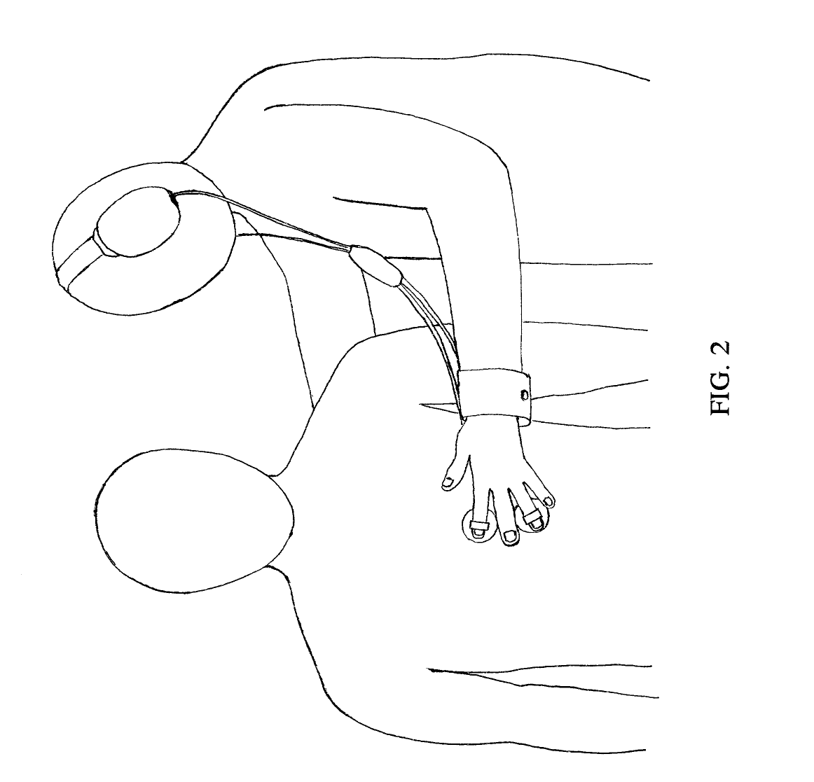

The two-headed focusing stethoscope (FIG. 2) described herein is the first stethoscope to address the problem of contaminating physiologic sounds. Even in the absence of environmental noise, sounds of laryngeal, bronchopulmonary, cardiovascular, and enteric origin often compete for the clinician's attention. The THFS amplifies the sound from the site of interest (e.g. the heart), while subtracting both physiologic and environmental sounds originating at other sites (e.g. breath, bowel, and room sounds).

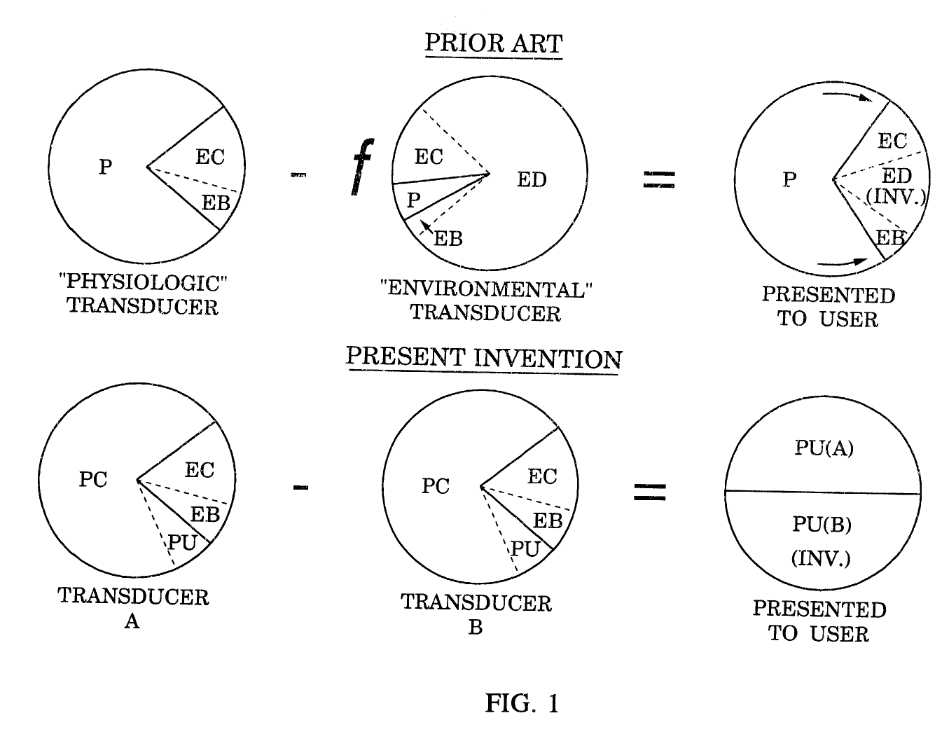

The THFS uses two identical transducers (A and B in FIG. 1), housed in identical chest pieces closed to the environment, positioned within a few inches of one another on the patient's body. Each transducer converts a combination of physiologic sound and contaminating environmental sound into electrical signals. The signal of transducer B is subtracted from the signal of transducer A, yielding a difference signal.

Since the chest pieces are positioned within a few inches of one another, the environmental sound which impinges upon one very nearly equals that which impinges upon the other. No such sound reaches the transducers directly, as the chest pieces are closed to the environment.

The chest pieces, furthermore, have identical sound transfer characteristics. The environmental sound which reaches transducer A through its chest piece (EC) and through the patient's body (EB) is therefore very nearly identical to that which reaches transducer B. Subtraction therefore eliminates essentially all environmental contamination.

Much of the physiologic sound transduced by the two proximate transducers is correlative (PC); a smaller fraction is unique (PU) to a given transducer. Subtraction eliminates the correlative fraction (PC) completely, yielding a difference signal comprised of the unique physiologic component of the signal transduced by transducer A [PU(A)], and an inverted version of the unique physiologic component of the signal transduced by transducer B [PU(B)(INV.)]. (When transduced by speakers, inverted signals produce sound which is perceptually indistinguishable from that produced by non-inverted signals.) Thus a focusing effect is achieved; sounds which originate close to either transducer are preserved, while others are attenuated.

### Theoretical Basis of the Invention

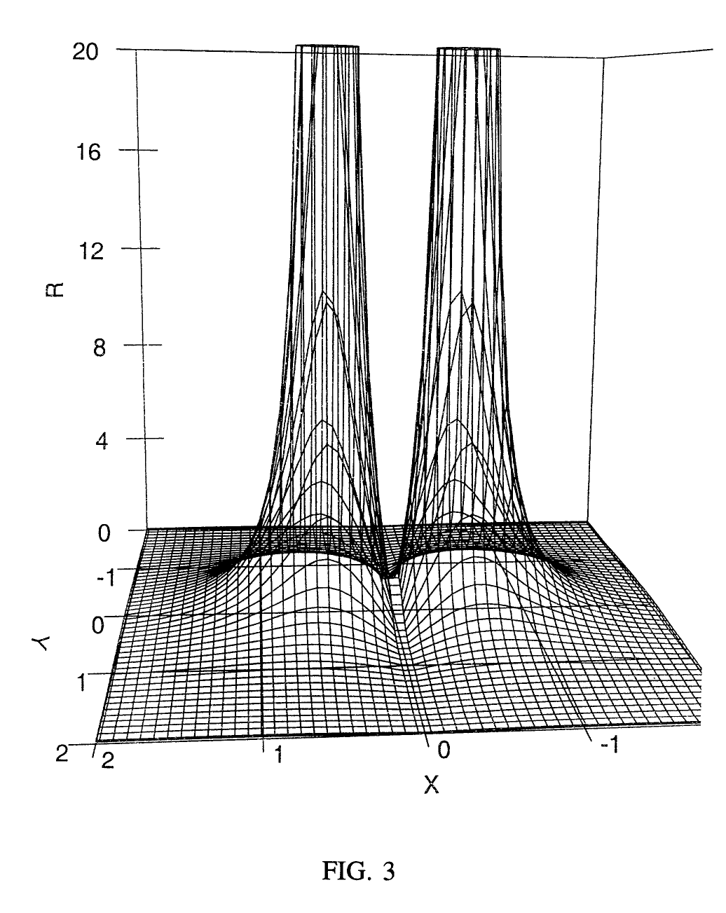

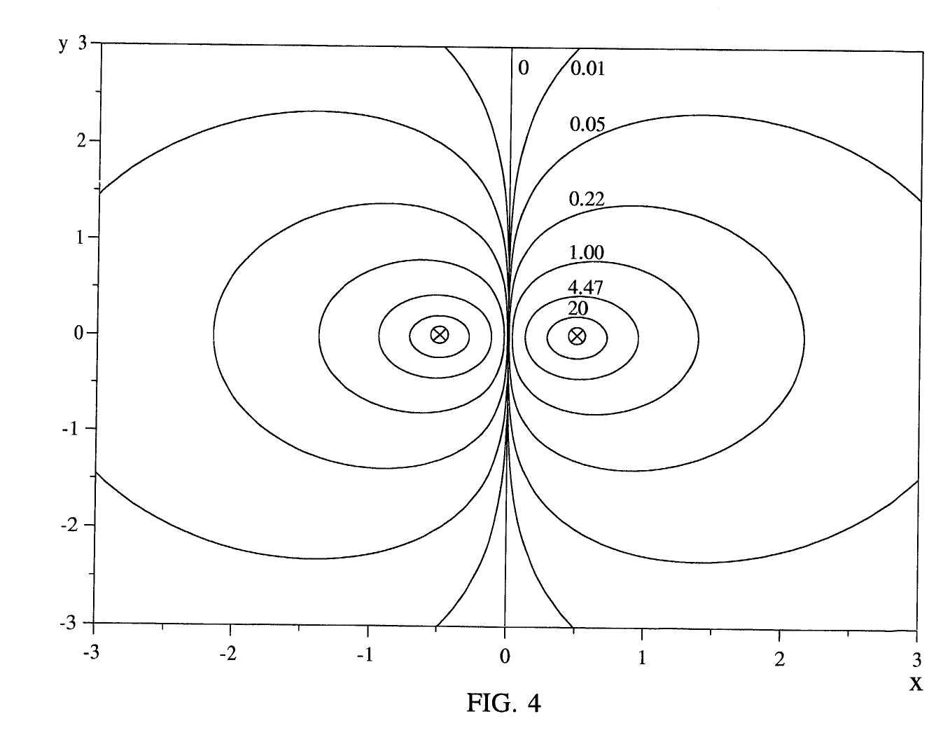

The distance d^{As} from transducer A, located at (x_{A},y_{A}), to a sound source S, located at (x_{S},y_{S}), is d_{AS}=[(x_{S}−x_{A})²+(y_{S}−y_{A})²]^{½}. The intensity of a sound at a transducer varies inversely with the square of its distance from the sound's source. The sound is therefore 1/(d_{AS})²=1/([(x_{S}−x_{A})²+(y_{S}−y_{A})²]^{½})²=1/[(x_{S}−x_{A})²+(y_{S}−y_{A})²] as intense at A as it is one unit from S. The difference between the sound's intensity at transducers A and B (x_{B},y_{B}) is 1/[(x_{S}−x_{A})²+(y_{S}−y_{A})²]−1/[(x_{S}−x_{B})²+(y_{S}−y_{B})²]. FIGS. 3 and 4 map the relative strengths of difference signals for sound sources of equal intensity positioned with respect to transducers A (\mathring{∘x} at −0.5,0) and B (\mathring{∘x} at +0.5,0) in a uniform medium. (The pattern is the same for any plane which includes line AB.)

For any given placement of transducers A and B in a uniform medium, both the absolute and relative differences between a sound's intensity at A and B decrease as its source moves away. The THFS exploits this to preserve sounds originating close to either transducer, while sharply attenuating both physiologic and environmental sounds originating farther away (FIGS. 3 and 4).

An exception to this distance effect applies to sounds which originate in the plane equidistant from A and B (the “midplane”, x=0 in FIGS. 3 and 4). These are equally intense (and in phase) at both transducers, and cancel one another completely during subtraction.

Virtually all environmental sound, and all physiologic sound common to both transducers, is eliminated (FIG. 1). The difference signal is comprised entirely of the unique component of signal A, and an inverted version of the unique component of signal B. In essence, the THFS focuses on sounds which originate close to either transducer, while eliminating those originating farther away, or in the midplane.

This focusing effect improves considerably on the attenuation of sound due to distance alone. For ease of calculation, consider first a sound originating from a source S at (x_{A}, y_{S}), when transducer A is at (x_{A}, 0) and B is at (x_{A+}1,0). The distance from the sound's origin to A is y_{S}, and its intensity (relative to its intensity one unit from S) at A is 1/yₛ². The distance to B is (1²+y_{S}²)^{½}, and its intensity at B is 1/(1+y_{S}²). The difference between the intensities at transducers A and B is 1/y_{S}²−1/(1+y_{S}²), which may be reduced to 1/[(y_{S}²)(1+Y_{S}²)].

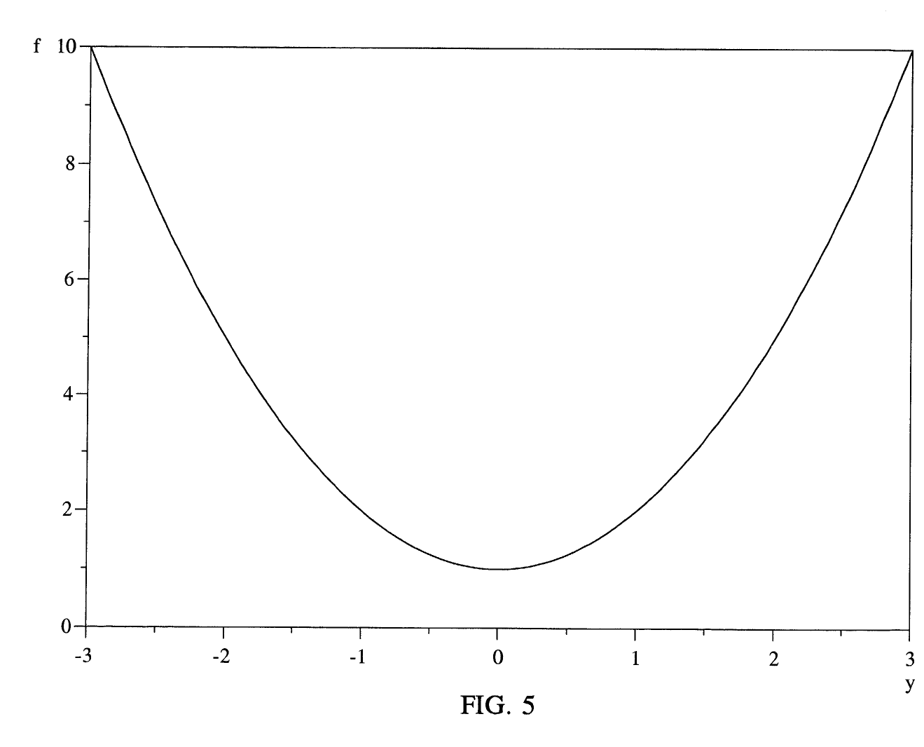

Dividing the intensity at A by the difference between the intensities at A and B yields 1+y_{S}². This ratio, of the attenuation of sound due to distance alone, to the THFS difference signal, is graphed in FIG. 5. Note that (when x_{S}=x_{A}) the THFS is 2, 5, and 10 times as efficient at attenuating sounds 1, 2, and 3 units, respectively, from transducer A as is distance alone.

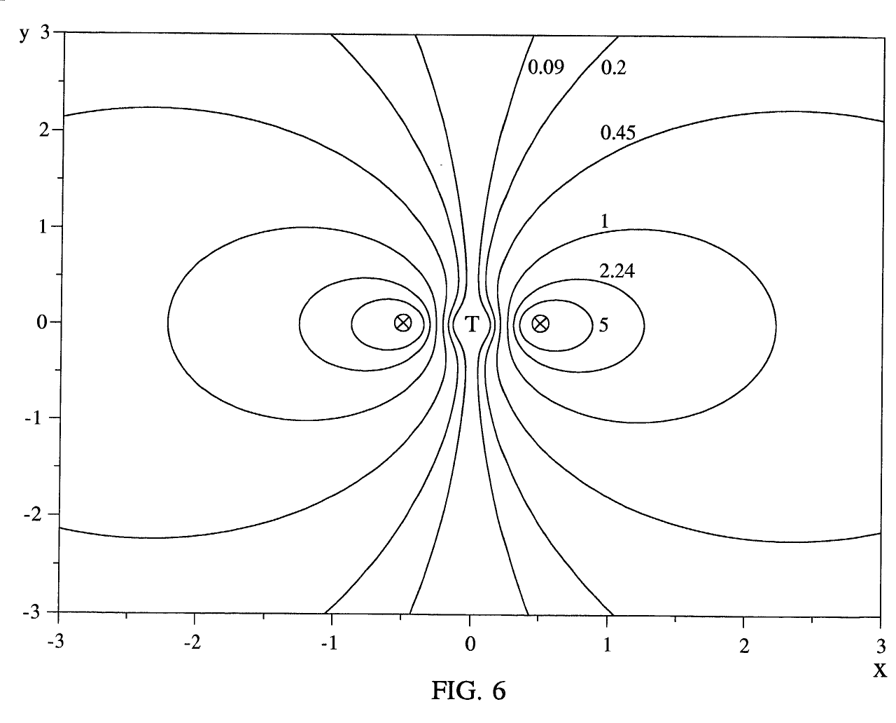

Calculations are more complex when all points in the XY plane are considered. In FIG. 6, the focusing effect of the THFS is compared to a reference single transducer (T) positioned midway between THFS transducers A (\mathring{∘x} at −0.5,0) and B (\mathring{∘x} at +0.5,0). Note that the THFS is both more efficient at preserving nearby sounds (factors>1) and attenuating distant sounds (factors<1), compared to the reference.

### Evaluation of Potential Problems

Phase shifts are a potential source of error. The speed of sound c in air at 72° F. is 1130 fps; in water at 98.6° F. it is 5069 fps. If sound originates a distance Δd feet closer to one transducer than the other, it will reach it Δt=Δd/c seconds sooner. For sources in the midplane (equidistant from transducers A and B), Δd=0 and Δt=0. Sounds which originate in the midplane therefore always reach both transducers in phase.

The surface of the human body is generally flat or convex, so physiologic sound sources are seldom in line with transducers A and B, and Δd is therefore less than the distance between them (Δd<d_{AB}). Furthermore, sound travels considerably faster in tissue (c≈5069 fps) than in air. For sounds of physiologic origin Δt, and the associated phase shifts, are therefore small. When the transducers are 4″ apart (d_{AB}=4″), Δd<4″, and Δt<(4/12)′/(5069 fps)=1/15207 second. A sound of >15207 Hz (the exact value depending on Δd) takes one cycle longer to reach the farther than the nearer transducer, arrives in phase, and is subtracted. A sound of half that frequency reaches the two transducers 180° out-of-phase, and is subtracted in antiphase. Subtraction of antiphase signals is equivalent to adding in-phase signals, and would therefore oppose the intent of subtraction. Sounds of 0 to 3802 Hz arrive less than 90° out-of-phase, and are subtracted in part. This subtraction band includes virtually all fundamental frequencies of physiologic origin. Sounds with wave lengths much greater than d always reach the transducers nearly in phase, and are subtracted almost completely. Virtually all normal and pathological heart sounds have wave lengths (in water at 98.6° F.) of 6 ⅓ feet or more (frequencies of 800 Hz or less), reach the transducers (positioned 4″ apart) less than 19° out-of-phase, and are subtracted efficiently.

The worst phase shifts involve sounds propagated through air (c=1130 fps) from environmental sources in line with transducers A and B (Δd=d_{AB}) If Δd=4″, then Δt=(4/12)′/(1130 fps)=1/3390 second. In this scenario, frequencies of (n)(3390 Hz) would reach the farther transducer n cycles later. When n is a whole number, the sound arrives in phase, and is subtracted in phase. When n is a whole number ±0.5, the sound arrives 180° out-of-phase, and is subtracted in antiphase (i.e. added). Alternating peaks of subtraction and addition result in a comb filter effect.

In practice, this “worst case” is barely realistic. Little sound is transmitted through the chest piece housings, which are closed to the environment. Most environmental sound is of relatively low frequency. The fundamental frequencies of a piano's notes, for example, range from 27.5 Hz to 4186 Hz. When the transducers are 4″ apart and the environmental sound source is in line with transducers A and B (worst case), 59% of these frequencies (27.5-847 Hz and 2543-4186 Hz) are subject to partial or complete subtraction, while only 41% (848-2542 Hz) are subject to partial or complete addition. Rotating the patient, when practicable, to bring the sound source into the midplane, reduces Δd to zero and results in complete subtraction at all frequencies.

The midplane effect can be used to dissect a composite of sounds into its components. The first heart sound S₁, for example, is comprised of the closure sounds of the tricuspid and mitral valves. If one of the THFS's chest pieces is placed directly over the tricuspid valve, and the other chest piece is positioned so that the mitral valve is equidistant from the two transducers (i.e. straddled by them), the sound of the mitral valve is subtracted from S₁. The procedure is reversed to subtract the sound of the tricuspid valve from S₁.

Thermal (Johnson) noise is discussed in Preferred Embodiment.

### Embodiments

Analog embodiments can generally be manufactured at lower cost than digital versions, and analog signal processing does not introduce quantization error. Digital models are easily interfaced with other digital devices, and exhibit better noise immunity than analog models. These and other considerations may influence which embodiment is ultimately marketed.

The embodiments described herein are just examples and not limitations. One skilled in the art will recognize that other embodiments are possible, consistent with the invention described.

### Simple Analog Version

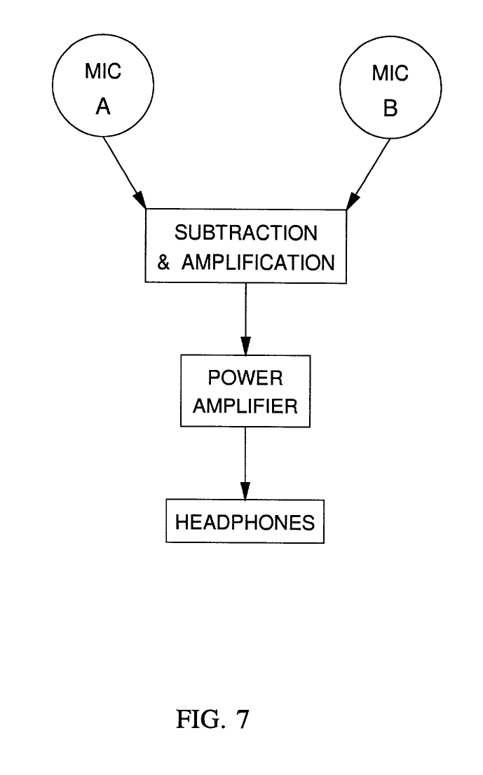

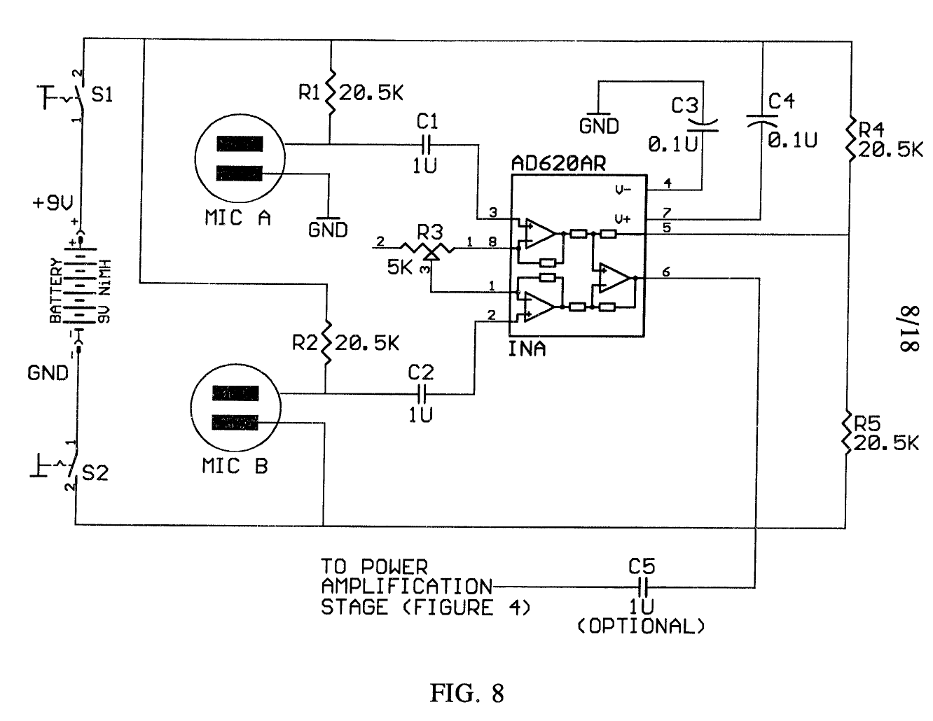

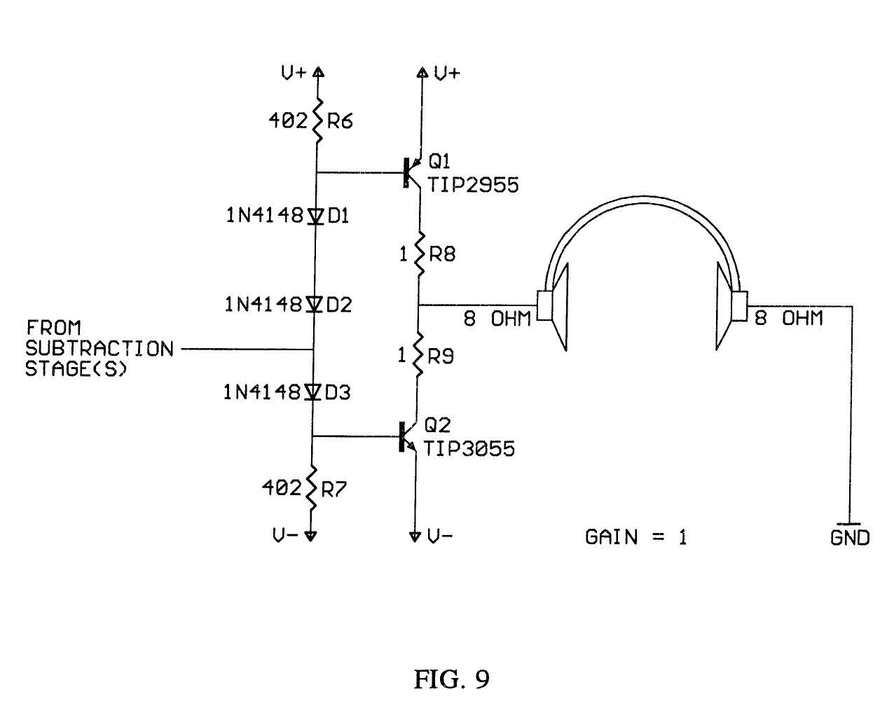

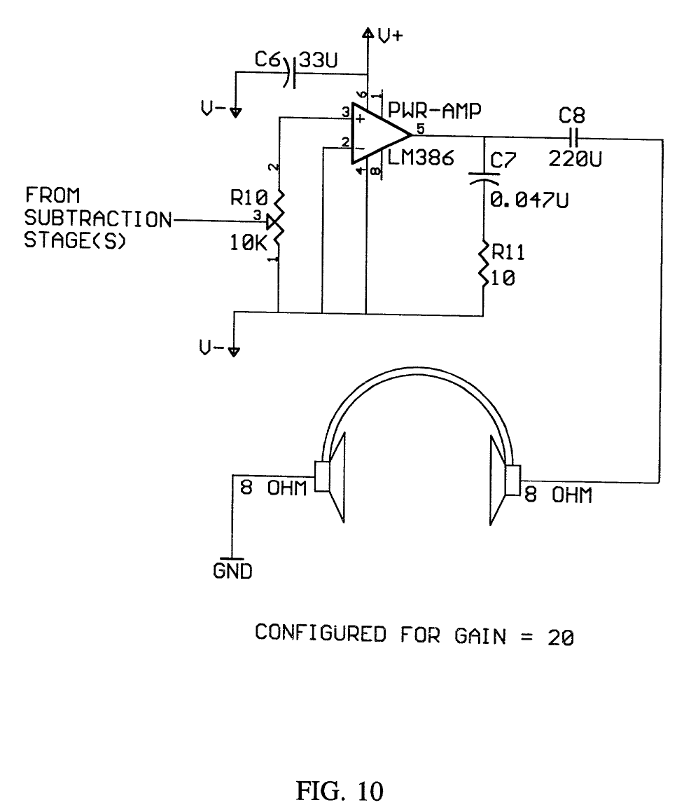

A simple analog version (FIG. 7) can be constructed with a single operational amplifier (configured as a subtractor) or an instrumentation amplifier (FIG. 8), and a power amplification stage (FIGS. 9 and 10). The instrumentation amplifier-based version diagrammed in FIG. 8 is considered first.

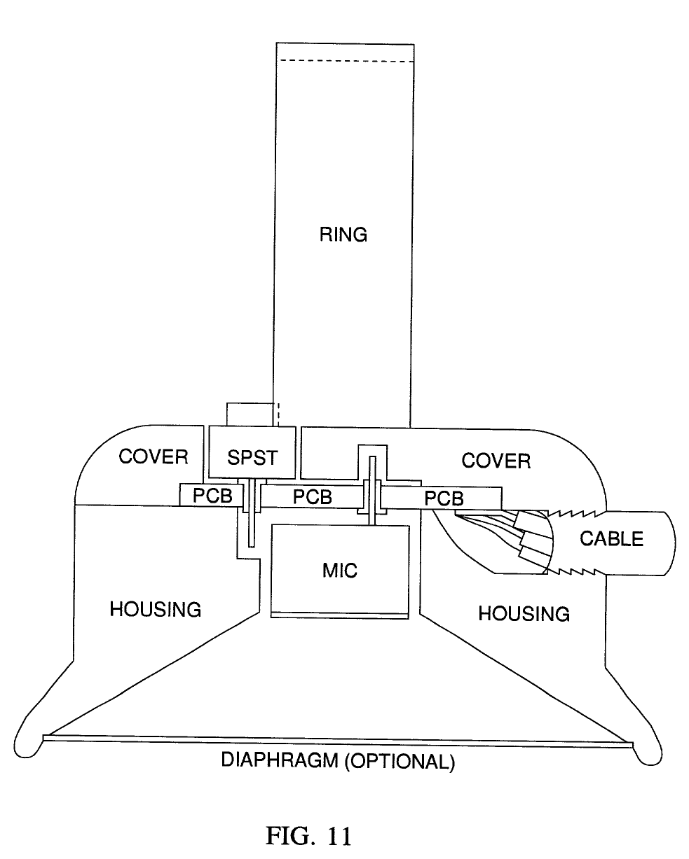

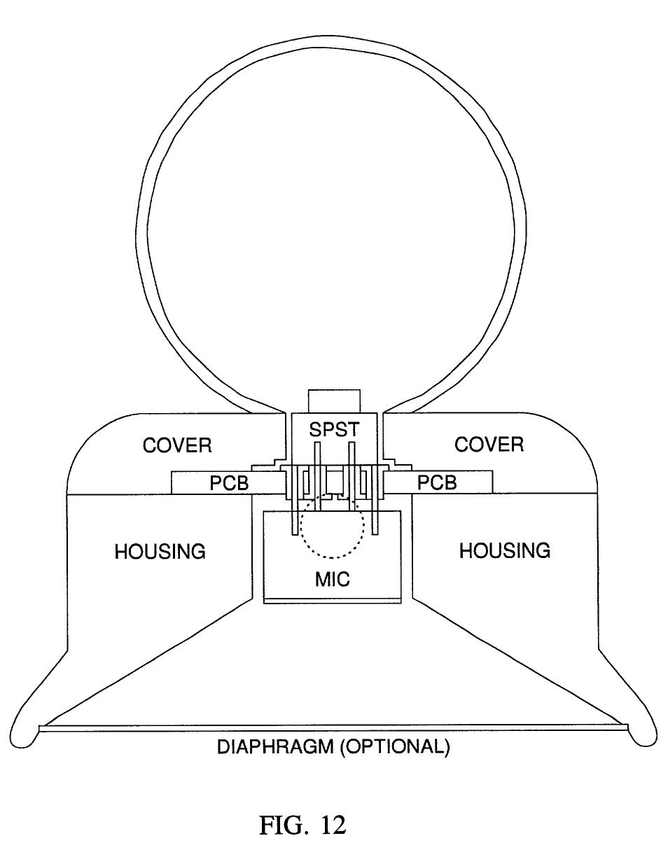

Power from a rechargeable 9-volt NiMH battery is conserved, as current flows only when both chest pieces (FIGS. 11 and 12), which function to hold the microphones, are pressed against the patient, closing momentary SPST switches S1 and S2 (FIG. 8). System voltage drops across resistors R1 and R2, providing phantom powering of appropriate voltage across the leads of capacitor microphones MIC A and MIC B. Capacitors C1 and C2 strip the signal from its DC bias. An instrumentation amplifier produces a difference signal of adjustable gain biased (through R4 and R5) to an appropriate voltage. This is output to a power amplification stage, and thence to headphones (FIGS. 9 and 10).

This simple version uses standard screened microphone cable (which functions to transmit electrical signals from the microphones to the subtraction circuitry), in which the screen forms the signal return path to ground. This allows some of the radiated interference which the screen is intended to drain to become superimposed on the signal. Since the difference signal is typically just ±15 μV, it is important to keep it free of contamination.

It is, furthermore, necessary to provide these small signals with adequate amplification; gains of 100,000 should be available. The simple version accomplishes this with acceptable high-frequency attenuation; the gain-bandwidth products of the AD620AR instrumentation amplifier and the LM386 power amplifier limit the attainable bandwidth to 7746 Hz.

Sounds of very low frequency (VLF) are often of diagnostic significance. In order to preserve VLF signal components, series coupling capacitance should be kept to a minimum. This can be accomplished by increasing the values, and reducing the number, of capacitors in the signal path.

### Preferred Embodiment

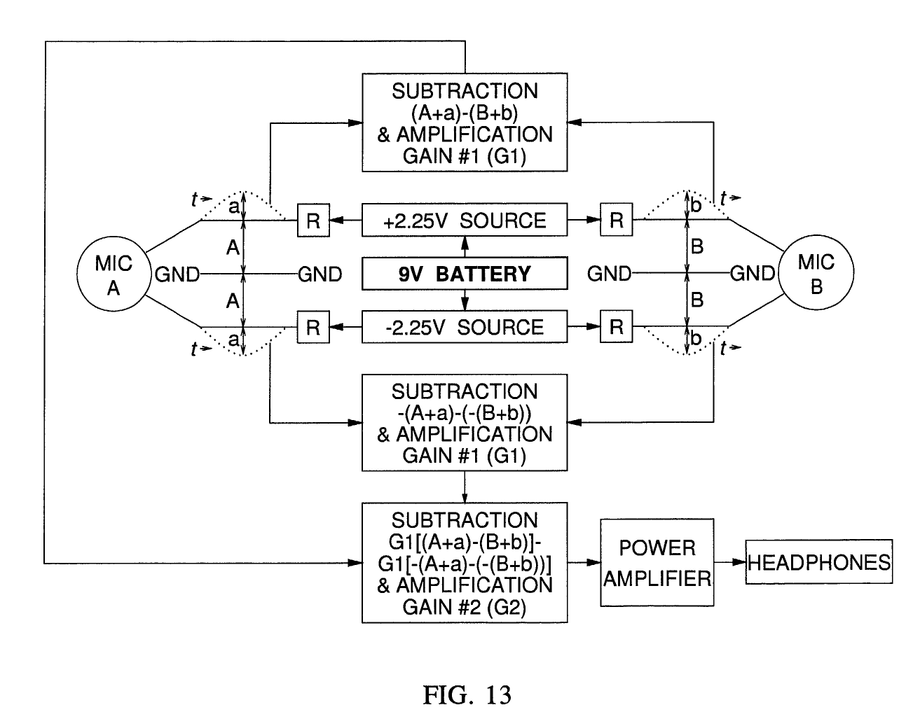

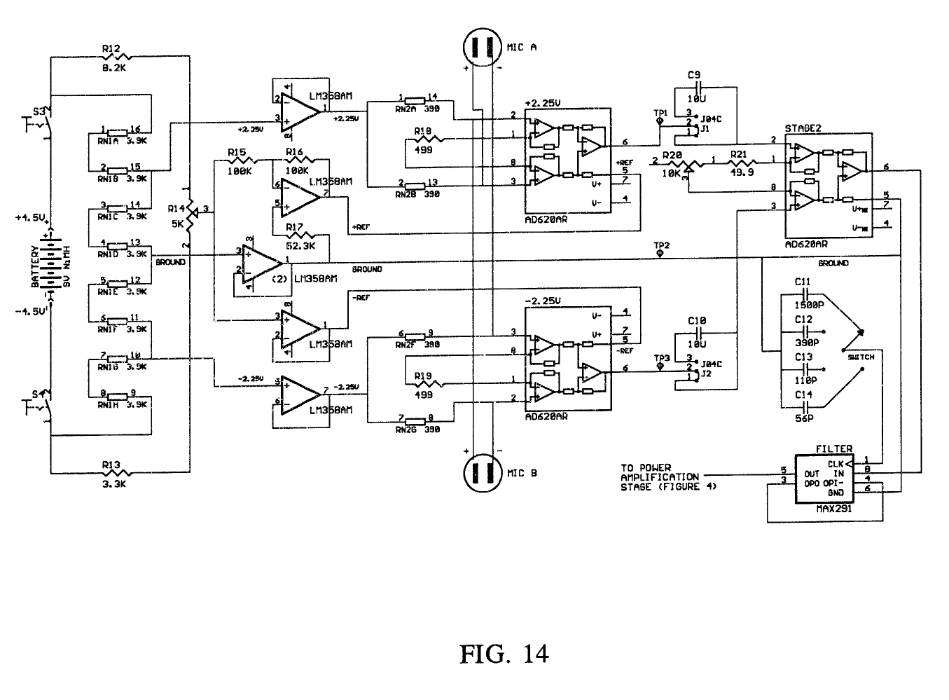

The preferred embodiment (FIGS. 13 and 14) addresses the shortcomings of the simple version. Balanced microphones enhance EMI rejection, and adjustable gains of 100,000 are available prior to the power amplification stage. VLF signal components are preserved, as no capacitors are required prior to filtering.

Current is supplied to the circuit by a 9-volt NiMH rechargeable battery only when both chest pieces (FIGS. 11 and 12), which function as holding means for the microphones, are pressed against the patient, closing a momentary-on light-touch SPST pushbutton switch (S3 and S4 in FIG. 14) in each. In addition to conserving battery power, this feature prevents contact noise from being amplified and transmitted to the user. The actuator for each switch may be located on the rim of the chest piece, between the diaphragm/bell portion of the chest piece and a cap section, or atop the chest piece (diagrammed).

Voltage division is accomplished by a resistor network (RN1 in FIG. 14), which supplies ≈+2.25, 0, and ≈−2.25-volt references to operational amplifiers configured as voltage followers. The followers provide the required voltage across the leads of sensitive (≈−60 dB) full-spectrum capacitor or back-electret microphones. Care is taken to eliminate radiated interference. Screened microphone cables, which serve to transmit electrical signals from the microphones to the subtraction circuitry, are grounded to a potential (GROUND) midway between those of the leads (≈+2.25 V and ≈−2.25 V); the voltage divider ensures that this relationship is maintained as the battery discharges. The signals from the positive and negative leads of each microphone are processed to eliminate common-mode interference and provide amplification.

The novel subtraction strategy employed by the THFS obviates the need for capacitors in the signal path. The signal from a balanced microphone is usually obtained by subtracting the instantaneous voltage of one lead from that of the other. Since the signals present in the two leads are inverted versions of one another (FIG. 13), they are subtracted in antiphase (added), and thus preserved. Interference, in contrast, affects the signals in both leads equally, and is eliminated.

In FIG. 13, A is the magnitude of the DC bias voltage, and a the magnitude of the signal voltage, for each lead of MIC A. Similarly, B is the magnitude of the DC bias voltage, and b the magnitude of the signal voltage, for each lead of MIC B. (For clarity, these signal components are not shown to scale; in reality, A>>a and B>>b.) Subtraction (not diagrammed) yields the voltage difference (A+a)−(−A−a)=2A+2a, which contains a large DC component 2A. A capacitor is used to pass only the signal 2a to the next stage. In a subtracting stethoscope, the signal 2b (stripped by another capacitor from (B+b)−(−B−b)=2B +2b) would then be subtracted from 2a, leaving 2 (a -b).

In contrast, the novel subtraction strategy employed by the preferred embodiment (FIG. 13) eliminates the need for capacitors in the signal path. If the impedances of MIC A and MIC B are equal, then the voltage drops across the resistors R are equal, and A=B. Since a and b “ride” on equal bias voltages, the instantaneous voltage of the positive wire of MIC B may be subtracted from that of MIC A, yielding the bias-free difference (A+a)−(B+b)=a−b. Subtracting the voltage of the negative wire of MIC B from that of MIC A similarly yields the bias-free difference −(A+a)−(−(B+b))=−a+b. These differences are then subtracted, leaving (a−b)−(−a+b)=2(a−b).

The two strategies produce the same result, but the first uses capacitors to remove the DC bias voltages, while the second removes them in the course of subtraction. Thus the second strategy eliminates the undesirable attenuation of important low-frequency signals attributable to series capacitance. Moreover, the intermediate values produced by the first strategy (2A+2a and 2B+2b, each typically including 4.2 V of DC bias) generally saturate amplifiers (when V+=4.5 V), even when no gain is applied. In contrast, a substantial gain (G1, fixed at 100 in FIG. 14), may be applied to the intermediate values (a−b and −a+b, each typically ±7.5 μV) produced by the second strategy. This reduces the number of stages required to reach the desired limiting gain of 100,000.

Unfortunately, the impedances Z_{MIC A} of MIC A and Z_{MIC B} of MIC B are seldom exactly equal. These impedances act as resistors in voltage dividers R−Z_{MIC A}−R and R−Z_{MIC B}−R between the +2.25 V and −2.25 V sources. Thus A≠B, and (A+a)−(B+b)≠a−b; a DC component equal to A−B contaminates the intermediate difference signal a−b. Similarly, a DC component equal to −A+B contaminates the intermediate difference signal −a+b.

Note that these DC components are always equal in magnitude and opposite in sign. In order to eliminate them, reference voltages, equal in magnitude and opposite in sign (+REF and −REF), are supplied to the +2.25 V and −2.25 V instrumentation amplifiers by unity-gain non-inverting and follower op-amps. These, in turn, receive their inputs from a potentiometer within a voltage divider. The potentiometer is adjusted until the DC biases of the instrumentation amplifier output signals converge. (If the adjustment drifts, jumpers J1 and J2 permit the user to perform a “quick fix”, passing the output signals through 10 μF capacitors C9 and C10 until the unit can be serviced.)

Thus the inputs to the STAGE 2 instrumentation amplifier have equal DC components, and subtraction leaves only the gain-adjusted signal component (G1)(2)(a−b)=200(a−b). This is still a low-voltage signal, and considerable additional gain (G2) may be applied. This is adjustable up to 1000 via logarithmic thumbwheel potentiometer R20 (FIG. 14).

Thermal (Johnson) noise results from random movements of atoms in conductors. The random nature of Johnson noise prevents its removal by common-mode rejection circuitry, and it is amplified along with the difference signal. Amplified Johnson noise is experienced as white noise.

The frequencies of normal heart sounds, pathologic heart sounds, physiologic fundamentals, and physiologic harmonics essentially all fall below 200, 800, 3000, and 6000 Hz, respectively. An 8th-order low-pass Butterworth filter (MAX291, FIG. 14) with corresponding thumbwheel switch-selectable (SWITCH, FIG. 14) corner frequencies removes 99, 96, 85, and 70 percent of Johnson noise, respectively, while preserving the sounds of interest.

The output of the filter may be input directly into the complementary push-pull transistor power amplification stage depicted in FIG. 9. (A capacitor is necessary only if the alternate LM386-based stage, depicted in FIG. 10, is used.) The signal from the power amplifier is used to power lightweight headphones with ear cups which substantially block environmental sound. The headphones feature full-spectrum speakers with large diaphragms to optimize the reproduction of low-frequency sounds.

FIGS. 10 and 14 depict the use of LM386 power amplifiers and LM358AM operational amplifiers (both manufactured by National Semiconductor Corporation of Arlington Tex.). FIGS. 8 and 14 depict the use of AD620AR instrumentation amplifiers (manufactured by Analog Devices Incorporated of Norwood Mass.). FIG. 14 also depicts the use of a MAX291 low-pass filter, manufactured by Maxim Integrated Products of Sunnyvale, Calif. One skilled in the art will recognize that other components may be substituted.

### Digital Version

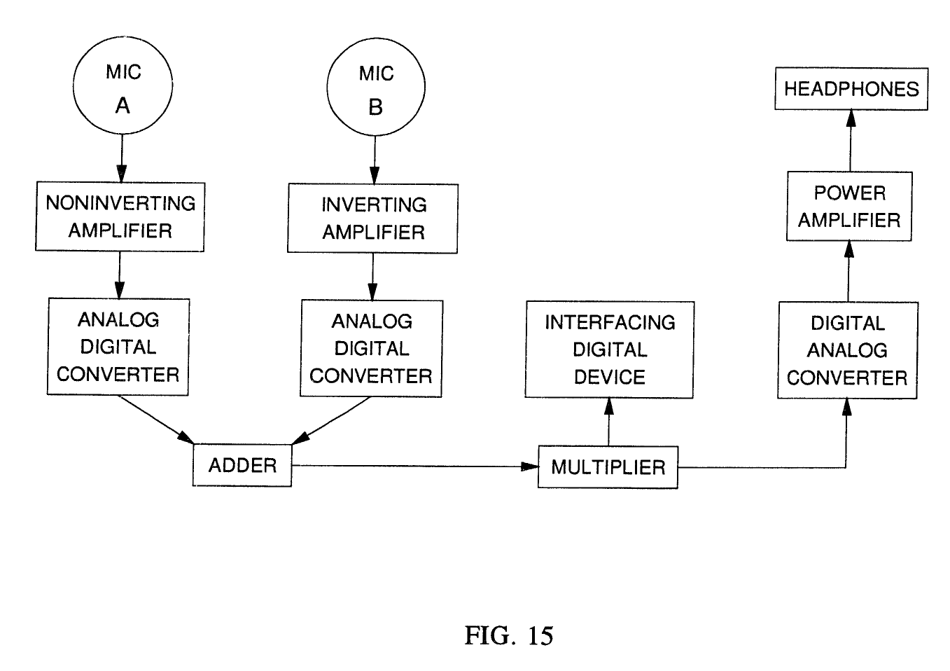

In the digital version (FIG. 15), the signals are taken from transducers A and B as in the simple analog version (FIGS. 7 and 8), and are processed by noninverting and inverting operational amplifiers, respectively. The amplifier outputs are digitized and added. The resulting difference signal may be multiplied, and/or output to another digital device (such as a computer) for further analysis. In any case, it is converted back to an analog signal by a digital-to-analog convertor (DAC), and then output to a power amplification stage (FIGS. 9 and 10).

A simple digital version can be constructed using a dual operational amplifier, a clock, a microcontroller, a DAC, and a power amplification stage. A suitable microcontroller is the PIC16C773, manufactured by Microchip Technology Incorporated of Chandler Ariz., but one skilled in the art will recognize that other models may be substituted.

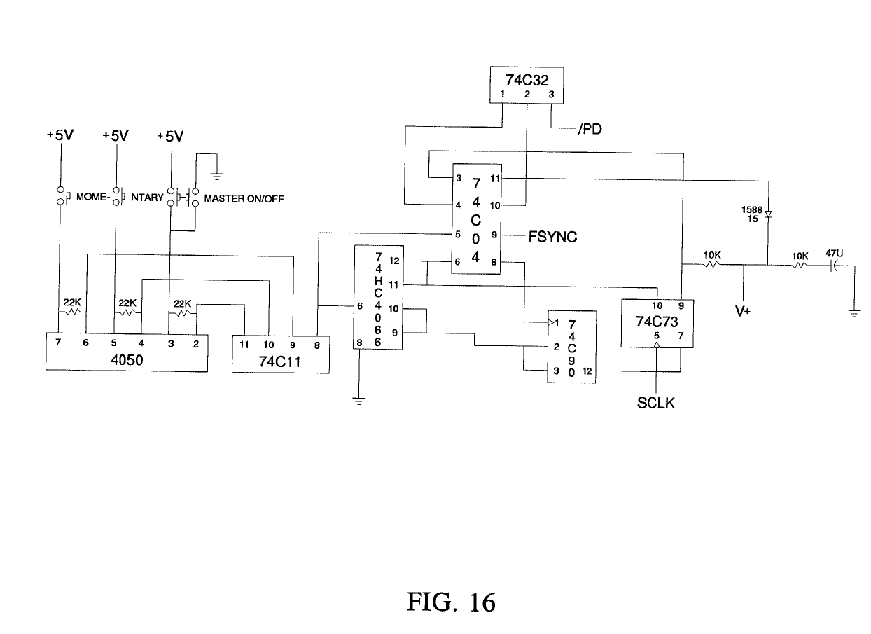

Although a microcontroller-based embodiment is preferred, a digital THFS may alternatively be constructed without any programmable components. One such embodiment (FIGS. 16, 17, and 18), which uses analog-to-digital (PCM1760 and DF1760) and digital-to-analog (PCM63P) convertors manufactured by Burr-Brown, a division of Texas Instruments Incorporated of Dallas Tex., is described. Common off-the-shelf ICs are identified by number only. As will be recognized, other components may be substituted.

### Power-Up

Debounced momentary-on light touch SPST pushbutton switches are closed when each chest piece (FIGS. 11 and 12) is pressed against the patient, activating the unit's power-up circuitry (FIG. 16). This preserves battery life, and eliminates the amplification of sound created when the chest pieces contact the skin.

The timing signals referred to in this section are described in more detail in the signal processing section which follows. The /PD input (pin 21) of the DF1760 must be held at ground for at least 2 sampling cycles after power is applied to the IC. It should then be held high. The accompanying schematic for power-up (FIG. 16) is designed to hold the /PD input at ground until the 5^{th} ground-to-positive transition of FSYNC.

When all three (the master on/off and both momentary-on) switches are closed, a high input on pin 6 of the 74HC4066 connects pin 9 to pin 8 (ground). This grounds both reset pins (2 & 3) of the 74C90, which is configured to provide a ground-to-positive transition at pin 12 on the 5^{th} high-to-ground transition of INV FSYNC. The 74C73 receives this signal at its J2 input (pin 7).

At the same time, the three closed switches result in a high input at pin 5 of the 74C04, and low input to pin 12 of the 74HC4066. This disconnects pins 10 and 11. The low is also seen at the K2 input (pin 10) of the 74C73. Both the J2 and K2 inputs are low (and clocking does nothing) until the pin 12 output of the 74C90 brings the 74C73's J2 input high. This makes the Q2 output (pin 9) go high on the next high-to-ground transition of SCLK. The remainder of the power-up circuit is taken from the PCM1760 data sheet (FIG. 13, page 13).

When either contact switch is opened, pin 6 of the 74HC4066 is brought low, and pin 9 is disconnected from pin 8 (ground). At the same time, pin 5 of the 74C04 is brought low, resulting in a high input at pin 12 of the 74HC4066. This connects the high at pin 11 with pin 10, resetting the 74C90, which provides a low input at pin 7 (J2) of the 74C73. Pin 10 (K2) of that IC is simultaneously brought high. On the next high-to-ground transition of SCLK, the output to pin 9 goes low.

### Signal Processing

In each chest piece (FIGS. 11 and 12), a large acoustic diaphragm improves transmission of physiologic sounds to a sensitive full-spectrum capacitor or back-electret microphone. The signal from MIC A (FIG. 15) is routed to a noninverting operational amplifier, and that from MIC B to an inverting operational amplifier, en situ (to prevent amplification of noise picked up during transmission). The amplified signals are then transmitted to the instrument's main electronics unit.

The connections are as diagrammed on the “Basic Connection Diagram of PCM1760 and DF1760” on page 7 of the technical data sheet. The signals are passed through low-pass filters to the PCM1760 analog-to-digital converter, then to the DF1760 digital decimating filter. Frequency of sampling (fs) is 48 KHz; the system clock frequency is 384 fs (18.432 MHz). The DF1760 is operated in “master mode” with “LSB first” (alternating 20-bit two's complement words representing LH and SH data co-sampled at 48 KHz) output.

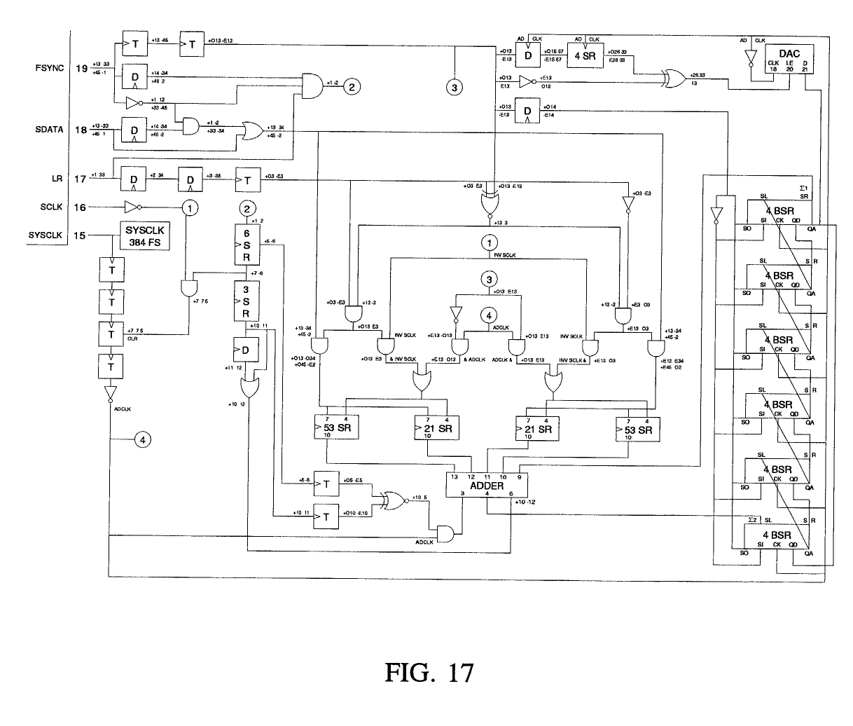

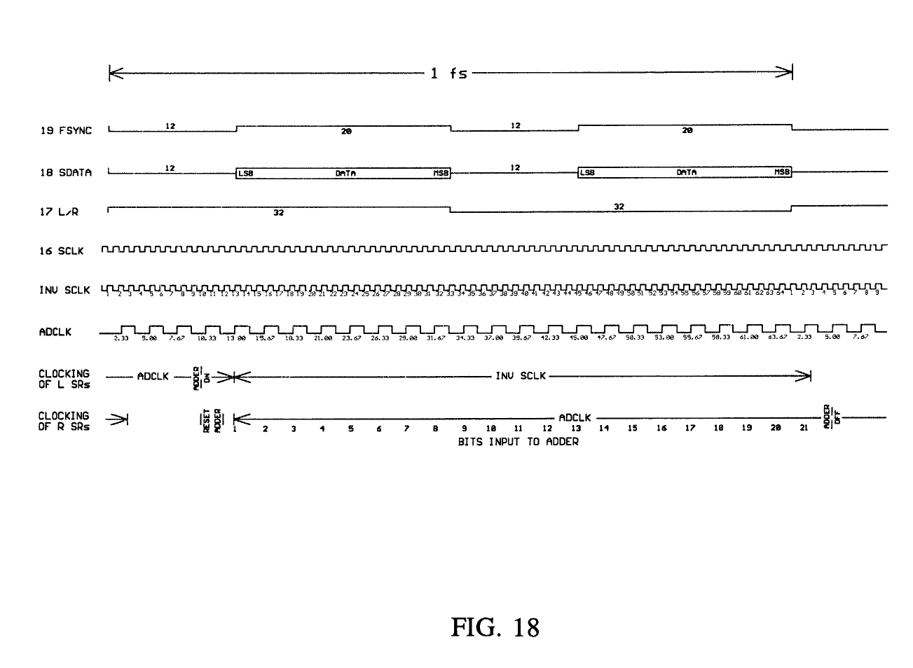

The logic diagram (FIG. 17) begins at the upper left with pins 16-19 of the DF1760. To avoid crowding, some electrical connections are indicated by circled like numerals (e.g. \mathring{∘1} is connected to \mathring{∘1}). The abbreviation SR in the logic diagram indicates a shift register, and BSR a bidirectional shift register; the number of stages precedes the abbreviation. Unless otherwise stated, all clocking is based on the inverted output of DF1760 pin 16 (INV SCLK), ground-to-positive transitions of which are numbered in FIG. 18 (The 53-stage and 21-stage shift registers are clocked by their inputs at pin 4.) For data lines, a number preceded by a plus sign indicates when data transmission begins, and one preceded by a minus sign when data transmission ceases. For timing lines, a positive number indicates when a signal goes high, and a negative number when it goes low. ‘O’ indicates an odd-numbered sample cycle, and ‘E’ an even-numbered sample cycle. Every clocked IC is clocked by ground-to-positive transitions of its clocking signal.

The main issue addressed is timing. The 4032 triple serial adder requires that both words be presented simultaneously, LSB first, with a sign bit following the MSB. Since the MSB of two's complement numbers is essentially a sign bit, I have repeated it by delaying it one clock, then ANDing it with an inverted FSYNC signal, and ORing the result with the original SDATA.

Since the 20-bit words from the LH and the SH appear at SDATA 32 SCLKs out-of-sync, they are sent (with the added sign bit) to 53 and 21-stage shift registers. The registers are clocked by SCLK (during the “loading” phase), from 13 through 2 (or, to 3). Word 1 is presented from 13 to 35, and word 2 from 45 to 3. [The 21-stage shift register presents Word 1, and 11 low bits, to the adder by the time the first bit of Word 1 has reached the output pin of the 53-stage shift register. During this time, leftover data is similarly out-put from the 53-stage register. Adder output resulting from this “data” is ignored, however, as it is presented to a bidirectional shift register while it is shifting toward the input pin.]

The first bits of Words 1 and 2 appear at the pin 10 outputs of their respective 53 and 21-stage shift registers at 3, simultaneous with cessation of the registers' INV SCLK clocking signals. In order to output the data to the 4032 adder, clocking must be switched to a signal compatible with its clock rate of 1.5 MHz (at 5 volts).

The 384 fs system clock is configured for a 96 fs output on pin 2. This replaces the first two Ts in the column of 4 Ts on the left of the logic diagram. Since it's important that SCLK and the 24 fs adder clock (ADCLK) maintain a predictable relationship, the third T on the chart (normally low from 6.33 to 7.67) is cleared once each fs from 7 to 7.5. ADCLK clocks the adder from 10 to 5. It is reset from 10 to 12, (receiving the requisite ground-to-positive and positive-to-ground ADCLK transitions at 10.33 and 11.67, respectively).

The 53 and 21-stage shift registers are clocked by ADCLK (during the “output” phase) for 1 fs, beginning (10 SCLKs after loading is completed) at 13. Bits are presented to the adder simultaneously on each of 21 ground-to-positive transitions of ADCLK from 13 to 2.33. Clocking of the registers continues, but clocking of the adder is discontinued prior to ADCLK's next ground-to-positive transition at 5. The 21 clockings allow registration of the added sign bit, and presentation of the sum of bits 20 to the bidirectional shift registers.

Since the adder must be fed slowly over nearly a full fs, two pairs of (53 and 21-stage) shift registers are required. One pair fills with words from odd-numbered samples, while the other feeds the adder with words from even-numbered samples. The pairs reverse their roles for data from the next sample.

Three 8-bit bidirectional shift registers are connected in series (in place of the six 4-bit BSRs diagrammed). They are clocked continuously by ADCLK, changing between shift right and shift left on 14 of each fs. Bits are input from the adder (LSB first) on 20 ADCLK clocks from 15.67 to 2.33. The final bit is repeated (as nonsense, after clocking of the adder is discontinued) 4 more times (on ADCLKs from 5 through 13). Then the direction of shift is reversed, and the four nonsense bits, followed by the 20 data bits (MSB first), are output to the PCM63P digital-to-analog convertor. It is necessary to ignore the added sign bit (bit 21), and use bidirectional shift registers to change from LSB first to MSB first, to satisfy the PCM63P's input requirements.

Continuously clocked by ADCLK, the PCM63P is enabled only from 26.33 to 13. It therefore ignores the nonsense bits coming from the BSRs from 15.67 to 23.67. The 20-bit data words are converted to analog signals. The schematic of FIG. 4, page 8 of the PCM63P's data sheet describes connections up to the power amplification stage (FIGS. 9 and 10).

### Operation

The operation of the THFS (FIG. 2) differs somewhat from that of the familiar acoustic stethoscope. The headphone cups are placed over the ears, and the index and ring fingers of one hand are placed through the rings of the chest pieces (FIGS. 11 and 12). The chest pieces are pressed against the patient over the area of interest, closing their integral SPST switches and powering the unit. (When the chest pieces are removed from the patient, the switches open and the unit is turned off.)

An appropriate filter corner frequency should be selected (SWITCH, FIG. 14) depending on the study to be performed:

(a.) 6000 Hz to assess the full spectrum of physiologic sounds. This setting may be necessary to further characterize a sound identified at a lower setting. All harmonics, which add tonal quality or timbre to sounds, are preserved. At this setting, however, the filter also passes 30 percent of Johnson noise.

(b.) 3000 Hz allows the clinician to hear virtually all physiologic fundamentals, while removing 85 percent of Johnson noise. Harmonics of higher frequencies, which add tonal quality to sounds, are eliminated. This is the preferred initial setting for general examination.

(c.) 800 Hz for cardiac auscultation. This setting passes both normal and abnormal heart sounds, while blocking 96 percent of Johnson noise.

(d.) 200 Hz to focus on low-frequency heart sounds. All normal heart sounds are passed, while 99 percent of Johnson noise is blocked. To prevent signal clipping, auscultation should generally begin with the volume thumbwheel (R20, FIG. 14) adjusted to a low setting. The volume is subsequently increased to an appropriate setting.

An understanding of FIGS. 3 and 4 figures importantly in the usage of the THFS. The focusing effect of the THFS is in inverse proportion to the distance between its microphones. To sharpen the stethoscope's focus, the chest pieces should be brought closer together.

Full advantage should be taken of the midplane effect. The chest pieces should be positioned so that the loudest physiologic sound source (e.g. the larynx of a crying pediatric patient) is in the midplane (i.e. equidistant from both transducers). The patient should be repositioned, if practicable, to bring loud environmental sound sources into the midplane as well. Sounds originating from either of two juxtaposed sound sources may be subtracted by placing the source to be subtracted in the midplane, and either transducer over the other source.

### Conclusion, Ramifications, and Scope of Invention

Thus the reader will see that the two-headed focusing stethoscope provides the user with a number of advantages over the prior art:

(a.) It attenuates contaminating environmental and physiologic sounds, making it easier for the user to focus his/her attention on a physiologic sound of interest.

(b.) It allows the user to assess the sounds of two juxtaposed sound sources, one at a time.

(c.) It preserves clinically-important sounds of very low frequency.

(d.) It conserves battery power, by powering the unit only when it is in use.

(e.) It prevents contact noise from being amplified and transmitted to the user.

(e.) Its circuitry is considerably less complex than the circuitry of existing noise-suppressing electronic stethoscopes.

While my above description contains many specificities, these should not be construed as limitations on the scope of the invention, but rather as exemplifications of a few possible embodiments thereof. Many other variations are possible.

Accordingly, the scope of the invention should be determined not by the embodiments illustrated, but by the appended claims and their legal equivalents.

I claim:

1. A method for preserving sounds originating close to a site under study, while sharply attenuating sounds originating farther away, comprising the steps of:
(a.) transducing all sounds present at said site, using two transducers which are functionally equivalent, placed close together at the site,
(b.) subtracting the signal of one said transducer from the signal of the other transducer, yielding a difference signal.
(c.) applying enough gain to said difference signal so that it can drive a speaker, and
(d.) transducing the amplified difference signal back to sound, whereby a focusing effect is achieved.

2. A method for subtracting sounds originating from a first sound source, from a composite of sounds originating from said first sound source and a second sound source, comprising the steps of:
(a.) positioning two functionally-equivalent transducers so that the first sound source is equidistant from said functionally-equivalent transducers, and said second sound source is closer to one of the transducers than the other transducer,
(b.) subtracting the signal of one transducer from the signal of the other transducer, yielding a difference signal.
(c.) applying enough gain to said difference signal so that it can drive a speaker, and
(d.) transducing the amplified difference signal back to sound, whereby a composite of sounds from two proximate sound sources can be dissected into its components.

3. A method for conserving battery power in an electronic stethoscope which has a chest piece, comprising closing said stethoscope's electronic circuitry only when said chest piece is placed against the object of study, whereby the battery drains only when the stethoscope is in use.

4. An electronic stethoscope, comprising:
(a.) first means for transducing sound into a first electrical signal,
(b.) second means for holding said first means, which permits the first means to be positioned as desired with respect to the object of study,
(c.) third means, which is functionally equivalent to the first means, for transducing sound into a second electrical signal,
(d.) fourth means, which is functionally equivalent to said second means, for holding said third means,
(e.) fifth means for subtracting said first electrical signal from said second electrical signal, yielding a difference signal,
(f.) sixth means for transmitting the electrical signals from the transducers to said fifth means,
(g.) seventh means for amplifying said difference signal, and
(h.) eighth means for transducing the amplified difference signal to sound.

5. The electronic stethoscope of claim 4, wherein the first means and the third means are microphones which are phantom-powered at potentials balanced about ground, and said sixth means is comprised of twisted-pair cables with grounded screens, whereby electromagnetic interference is minimized and its rejection is enhanced.

6. The electronic stethoscope of claim 4, wherein the signal processing strategy employed by said fifth means, for subtracting the first electrical signal from the second electrical signal, removes direct current bias in the course of subtraction, whereby no capacitors are required in the signal path, and attenuation of low-frequency signals due to series capacitance is eliminated.

7. The electronic stethoscope of claim 4, wherein the fifth means uses analog signal processing to accomplish the required subtraction.

8. The electronic stethoscope of claim 4, wherein the fifth means uses digital signal processing to accomplish the required subtraction.

9. The electronic stethoscope of claim 4, further including a low-pass filtering means, the input of which is connected to the output of the seventh means, and the output of which is connected to the eighth means; the corner frequency of which is selected to preserve frequencies of clinical significance, while attenuating amplified Johnson noise of higher frequencies.

10. An electronic stethoscope, comprising:
(a.) two microphones which are functionally equivalent to one another,
(b.) a rigid housing for each said microphone, small enough to be readily manipulated with thumb and forefinger, having a socket to accommodate the microphone, and a funnel which widens outward from said socket, the combination of said housing and its contained microphone being a chest piece,
(c.) a battery-powered electronic circuit, having two inputs and an output, configured to amplify the difference in voltage between said inputs,
(d.) a cable, connected at one end to one of the inputs, and at the other end to one of the microphones,
(e.) a second cable, connected at one end to the other input, and at the other end to the other microphone, and
(f.) a speaker, connected to said output of said battery-powered electronic circuit by
(g.) a third cable, whereby the difference between the sounds transduced by the two microphones is amplified and presented to the user.

11. The electronic stethoscope of claim 10, wherein the microphones are phantom-powered at potentials balanced about ground, and the cables are of the twisted-pair variety with grounded screens, whereby electromagnetic interference is minimized, and its rejection is enhanced.

12. The electronic stethoscope of claim 10, further including, in at least one of said chest pieces, a momentary-on switch, positioned such that it is closed when the chest piece is pressed against the object of study, and connected within the battery-powered electronic circuit such that battery power is supplied to the circuit only when the switch is closed, whereby the battery drains only when the stethoscope is in use.

13. The electronic stethoscope of claim 10, wherein the signal processing strategy, employed by the battery-powered electronic circuit, removes direct current bias in the course of measuring the difference in voltage between the inputs, whereby no capacitors are required in the signal path, and attenuation of low-frequency signals due to series capacitance is eliminated.

14. The electronic stethoscope of claim 10, wherein the difference in voltage between the inputs of the battery-powered electronic circuit is determined and amplified using analog signal processing techniques.

15. The electronic stethoscope of claim 10, wherein the difference in voltage between the inputs of the battery-powered electronic circuit is determined and amplified using digital signal processing techniques.

16. The electronic stethoscope of claim 10, further including a low-pass filter, the input of which is connected to the output of the battery-powered electronic circuit, and the output of which is connected to said speaker, the corner frequency of which is selected to preserve frequencies of clinical significance, while attenuating amplified Johnson noise of higher frequencies.

17. The electronic stethoscope of claim 10, wherein said speaker has a cone which is considerably larger than that of any earphone speaker, whereby lower frequency sounds can be faithfully reproduced, compared to those reproduced by earphone speakers.
